(19) United States
(12) Patent Application Publication
(10) Pub. No.: US 2026/0232391 A1
(43) Pub. Date: Aug. 13, 2026

(54) SURGICAL ROBOTIC SYSTEM WITH INTEGRATED ELECTROSURGICAL UNIT IN A SURGICAL INSTRUMENT

(71) Applicant: Covidien LP, Mansfield, MA (US)

(72) Inventors: Dylan R. Kingsley, Broomfield, CO (US); Zachary S. Heiliger, Nederland, CO (US); Christopher T. Tschudy, Arvada, CO (US); Daniel A. Joseph, Golden, CO (US); Haralambos P. Apostolopoulos, Westminster, CO (US)

(21) Appl. No.: 19/155,403

(22) PCT Filed: Feb. 6, 2024
(86) PCT No.: PCT/IB2024/051097

§ 371 Date: Aug. 11, 2025

Related U.S. Application Data
(60) Provisional application No. 63/445,324, filed on Feb. 14, 2023.

(51) Int. Cl. A61B 34/30; A61B 17/00; A61B 18/12

(52) U.S. Cl. CPC A61B 34/30; A61B 18/1206; A61B 2017/00398; A61B 2017/00477

(57) ABSTRACT

A surgical robotic system includes an instrument drive unit having at least one motor and at least one power supply contact. The system also includes an electrosurgical generator configured to couple to the instrument drive unit. The electrosurgical generator includes a generator circuit configured to couple to the at least one power supply contact and to generate electrosurgical energy. The system further includes an electrosurgical instrument configured to couple to the instrument drive unit and the electrosurgical generator. The electrosurgical instrument is actuatable by the instrument drive unit and energizable by the electrosurgical generator.

### CROSS-REFERENCE TO RELATED APPLICATIONS

This application claims the benefit of U.S. Provisional Patent Application Ser. No. 63/445,324, filed Feb. 14, 2023, the entire content of which is incorporated herein by reference.

### BACKGROUND

Surgical robotic systems are used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. Surgical robotic systems are used with a variety of electrosurgical instruments that are currently powered using conventional generators located outside a sterile barrier. Remote location of the electrosurgical generators requires extending and routing lengthy wires conducting electrosurgical energy to the instrument.

### SUMMARY

According to one embodiment of the present disclosure, a surgical robotic system is disclosed. The surgical robotic system includes an instrument drive unit having at least one motor and at least one power supply contact. The system also includes an electrosurgical generator configured to couple to the instrument drive unit. The electrosurgical generator includes a generator circuit configured to couple to the at least one power supply contact and to generate electrosurgical energy. The system further includes an electrosurgical instrument configured to couple to the instrument drive unit and the electrosurgical generator. The electrosurgical instrument is actuatable by the instrument drive unit and energizable by the electrosurgical generator.

Implementations of the above embodiment may include one or more of the following features. According to one aspect of the above embodiment, the electrosurgical instrument may be a monopolar scissors, an electrocautery hook, an electrocautery spatula, an electrocautery blade, a bipolar forceps, or a vessel sealer. The electrosurgical instrument may include at least one instrument coupler configured to engage the at least one motor. The electrosurgical generator may include at least one generator coupler configured to engage the at least one motor and the at least one instrument coupler. The instrument drive unit may further include a plurality of first communication contacts, the electrosurgical generator may include a plurality of generator passthrough communication contacts, and the electrosurgical instrument may include a plurality of second communication contacts, such that the electrosurgical instrument is in electrical communication with the instrument drive unit. The surgical robotic system may include a sterile interface module configured to couple the instrument drive unit to the electrosurgical generator. The sterile interface module may include at least one interface coupler configured to interconnect the at least one generator coupler and the at least one instrument coupler. The sterile interface module may include a plurality of interface passthrough communication contacts configured to provide electrical communication between the instrument drive unit and the electrosurgical instrument through the sterile interface and electrosurgical generator. The generator circuit may be a current source or a voltage source. The surgical robotic system may include a robotic arm configured to support the instrument drive unit and the electrosurgical instrument.

According to another embodiment of the present disclosure, a surgical robotic system is disclosed. The surgical robotic system includes an instrument drive unit having at least one motor and at least one power supply contact. The system also includes an electrosurgical instrument having an instrument housing configured to couple to the instrument drive unit. The electrosurgical instrument is actuatable by the instrument drive unit. The system also includes an electrosurgical generator configured to couple to the instrument housing of the electrosurgical instrument and to receive electrical input power from the at least one power supply contact through the electrosurgical instrument. The electrosurgical generator includes a generator circuit configured to generate electrosurgical energy from the input power to energize the electrosurgical instrument.

Implementations of the above embodiment may include one or more of the following features. According to one aspect of the above embodiment, the electrosurgical instrument may be a monopolar scissors, an electrocautery hook, an electrocautery spatula, an electrocautery blade, a bipolar forceps, or a vessel sealer. The electrosurgical instrument may include at least one instrument coupler configured to engage the at least one motor. The electrosurgical generator may include a generator housing having a proximal plate configured to be disposed between the instrument drive unit and the electrosurgical instrument. The proximal plate may include at least one opening to allow for engagement between at least one instrument coupler and the at least one motor. The surgical robotic system may include a robotic arm configured to support the instrument drive unit and the electrosurgical instrument. The generator circuit may be a current source or a voltage source. The instrument drive unit may further include a plurality of first communication contacts and the electrosurgical instrument may include a plurality of second communication contacts, such that the electrosurgical instrument is in electrical communication with the instrument drive unit. The electrosurgical generator may include a plurality of teeth extending from the generator housing and configured to engage the instrument housing.

According to a further embodiment of the present disclosure, a surgical robotic system is disclosed. The surgical robotic system includes an instrument drive unit having a plurality of motors, a plurality of power supply contacts, and a plurality of communication contacts. The system also includes an electrosurgical generator configured to couple to the instrument drive unit. The electrosurgical generator includes a generator circuit configured to couple to the plurality of power supply contacts and to generate electrosurgical energy through a plurality of output power contacts. The generator also includes a plurality of generator couplers configured to engage the plurality of motors and a plurality of generator passthrough communication contacts. The system further includes an electrosurgical instrument configured to couple to the electrosurgical generator. The electrosurgical instrument is actuatable by the instrument drive unit and is energizable by the electrosurgical generator. The electrosurgical instrument also includes a plurality of electrosurgical contacts configured to electrically couple to the plurality of output power contacts and a plurality of instrument couplers configured to engage the plurality of generator couplers.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a side view of an integrated electrosurgical generator, the instrument drive unit, and the surgical instrument disconnected from each other according to an embodiment of the present disclosure;

FIG. 8 is a side view of the electrosurgical generator of FIG. 7, the instrument drive unit, and the surgical instrument connected to each other;

FIG. 9 is a perspective view of a sterile interface module according to an embodiment of the present disclosure;

FIG. 10 is a partial, cross-sectional view of the electrosurgical generator of FIG. 7 and the surgical instrument being coupled to the sterile interface module;

FIG. 11 is a schematic diagram of the electrosurgical generator according to an embodiment of the present disclosure;

FIGS. 12A-C show an electrosurgical generator and the surgical instrument according to another embodiment of the present disclosure;

FIGS. 13A and 13B show the electrosurgical generator of FIGS. 12A-C and the surgical instrument coupled to the instrument drive unit; and

FIGS. 14A and 14B show an electrosurgical generator and the surgical instrument according to a further embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands to control one or more actuators of the robotic arm, which would, in turn, move the robotic arm and the instrument in response to the movement commands.

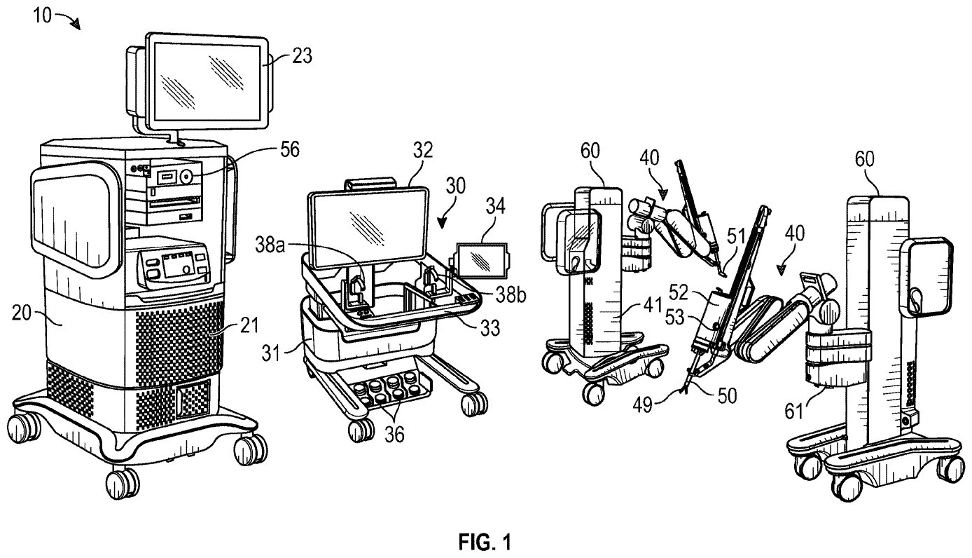

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left, and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

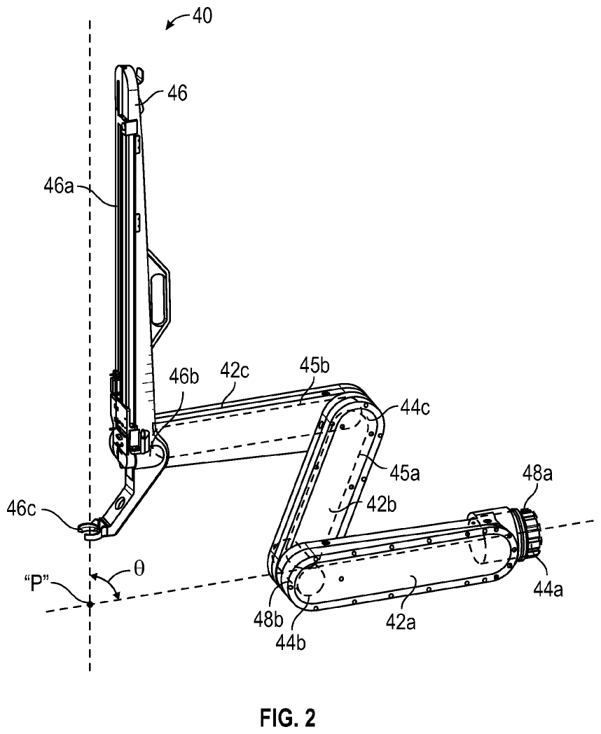

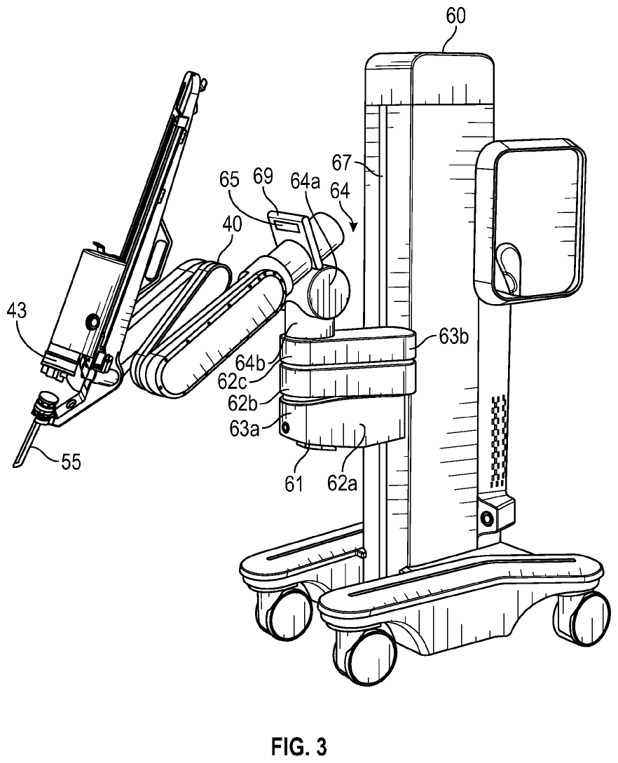

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48 b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some, or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and is configured to receive an instrument drive unit (IDU) 52 (FIG. 1). The IDU 52 is configured to couple to an actuation mechanism of the surgical instrument 50 and/or the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

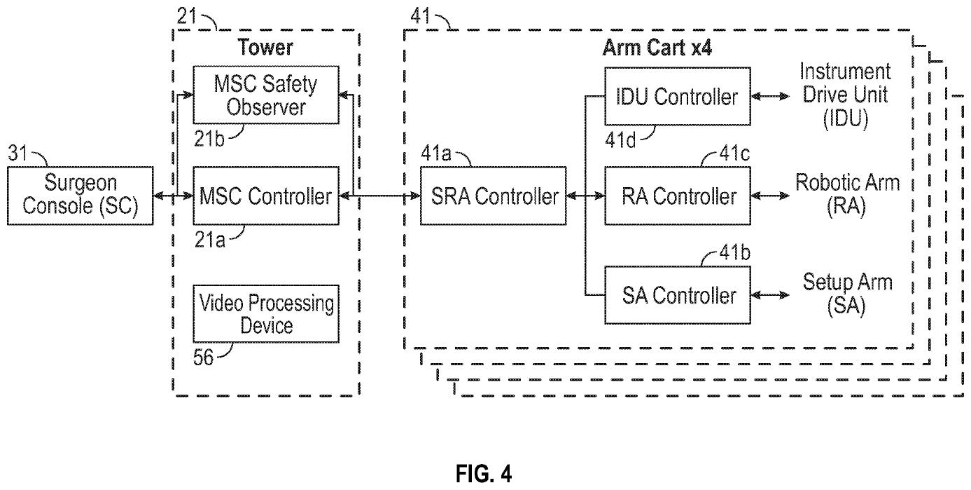

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41 a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

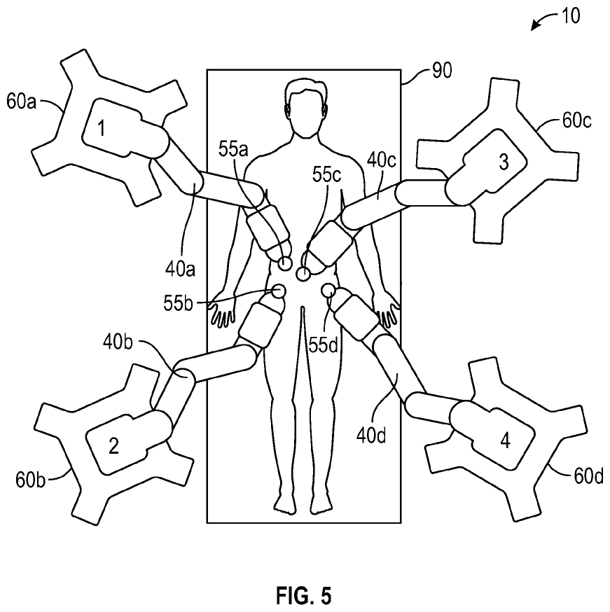

With reference to FIG. 5, the surgical robotic system 10 is setup around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

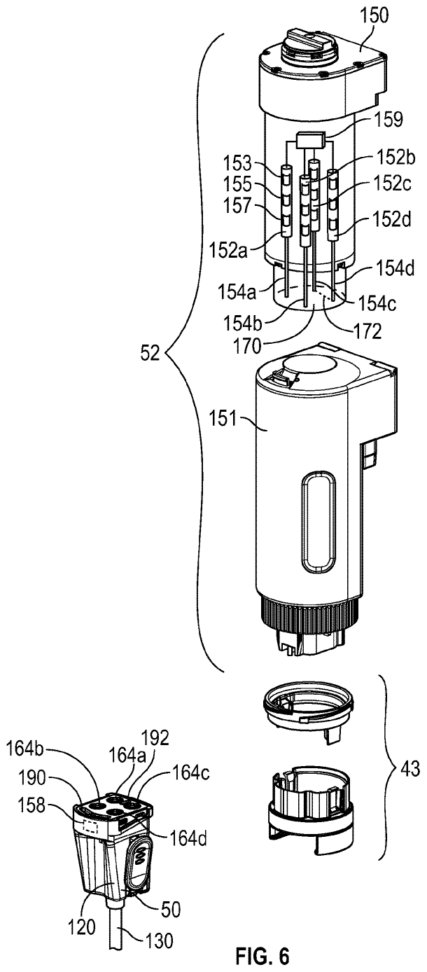

With reference to FIG. 6, the IDU 52 is shown in more detail and is configured to transfer power and actuation forces from its motors 152a-d to the instrument 50 to drive movement of components of the instrument 50, such as articulation, rotation, pitch, yaw, clamping, cutting, etc. The IDU 52 is also be configured to energize the instrument 50, which is an electrosurgical instrument, which may include, but not limited to, monopolar scissors, electrocautery hook, electrocautery spatula, electrocautery blade, bipolar forceps, and vessel sealer.

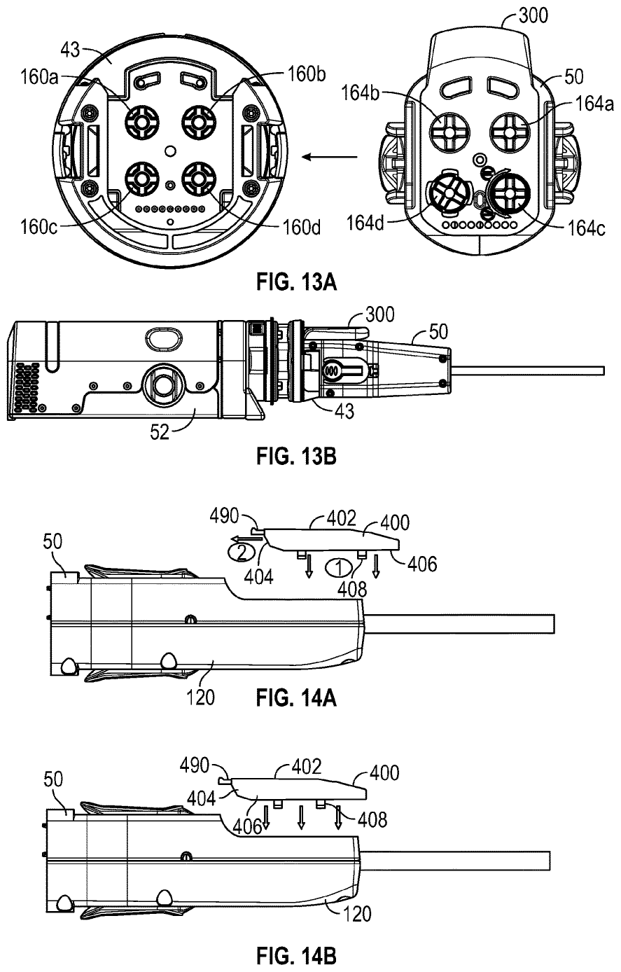

The IDU 52 includes a motor pack 150 and a sterile barrier housing 151. Motor pack 150 includes motors 152a-d for controlling various operations of the instrument 50. The instrument 50 is removably couplable to IDU 52. As the motors 152a-d of the motor pack 150 are actuated, rotation of the drive transfer shafts 154a, 154b, 154c, 154d of the motors 152a-d, respectively, is transferred to the drive assemblies of the instrument 50. The instrument 50 is configured to transfer rotational forces/movement supplied by the IDU 52 (e.g., via the motors 152a-d of the motor pack 150) into longitudinal movement or translation of the cables or drive shafts to effect various functions of an end effector assembly 140 (FIG. 13C).

Each of the motors 152a-d includes a current sensor 153, a torque sensor 155, and an encoder sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder 157 may be any device that provides a sensor signal indicative of the number of rotations of the motor 152a, such as a mechanical encoder or an optical encoder. Parameters which are measured and/or determined by the encoder 157 may include speed, distance, revolutions per minute, position, and the like. The sensor signals from sensors 153, 155, 157 are transmitted to the IDU controller 41d, which then controls the motors 152a-d based on the sensor signals. In particular, the motors 152a-d are controlled by an actuator controller 159, which controls torque outputted and angular velocity of the motors 152a-d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

The instrument 50 includes the housing 120, a shaft 130 extending distally from housing 120, and end effector assembly (not shown) extending distally from shaft 130. Housing 120 of instrument 50 is configured to selectively couple to IDU 52 of robotic, to enable motors 152a-d of IDU 52 to operate the end effector assembly 140 of the instrument 50. Housing 120 of instrument 50 supports a drive assembly that is mechanically actuated by the motors 152a-d of the IDU 52.

The surgical instrument 50 also includes a storage device 158 having non-volatile storage medium (e.g., EEPROM) that is configured to store any data pertaining to the surgical instrument 50, including but not limited to, usage count, identification information, model number, serial number, calibration data, and the like. In embodiments, the data may be encrypted and is only decryptable by the IDU controller 41d. The data may also be used by the IDU controller 41d to authenticate the surgical instrument 50. The storage device 158 may be configured in read only or read/write modes, allowing the IDU controller 41d to read as well as write data onto the storage device 158.

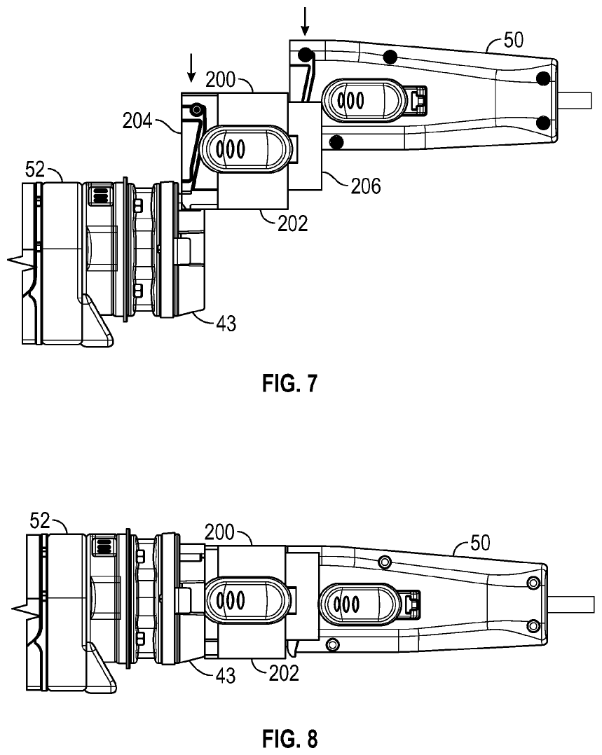

With reference to FIGS. 7-10, the system 10 includes a portable electrosurgical generator 200 configured to be coupled to the instrument 50 and to receive electrical power, e.g., DC power, from the IDU 52. This configuration avoids having an electrosurgical generator being disposed remotely from the robotic arm 40, e.g., at the control tower 20, and to extend cables from the generator to the instrument 50.

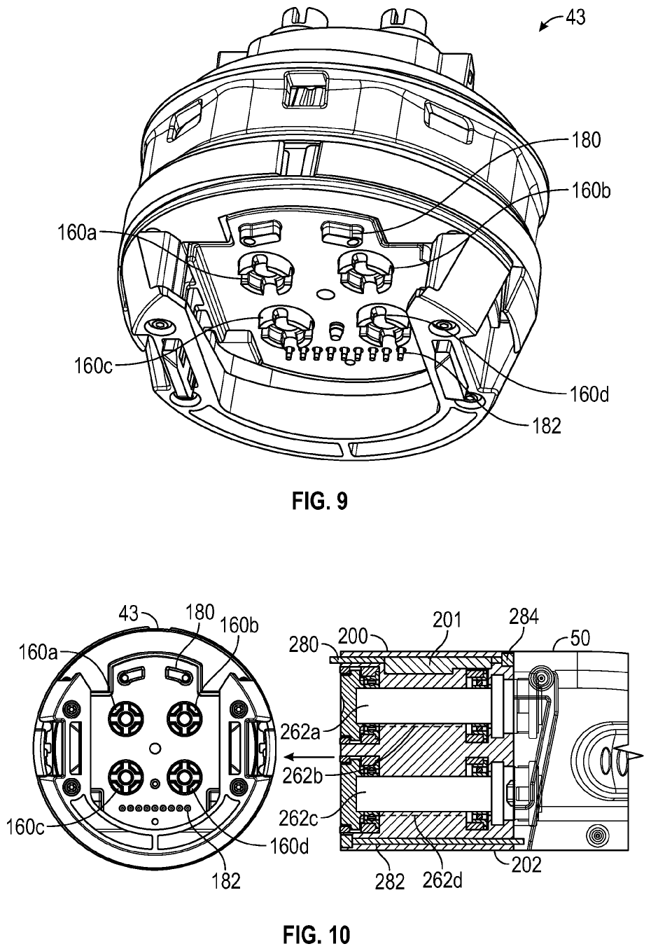

The electrosurgical generator 200 includes a housing 202 having a distal face 204 configured to engage with the SIM 43 and a proximal face 206 configured to engage the instrument 50 as shown in FIGS. 7 and 8. In particular, the instrument 50 includes a plurality of couplers 164a-d (FIG. 6), which are actuated by the motors 152a-d. The motors 152a-d engage the couplers 164a-d through the SIM 43, which also includes passthrough couplers 160a-d (FIG. 9). Similarly, the generator 200 includes generator couplers 262a-d (FIG. 10). Thus, the motors 152a-d of the IDU 52 engage the couplers 160a-d of the SIM 43, which in turn, engage the generator couplers 262a-d, and finally the couplers 164a-d of the instrument 50.

With reference to FIGS. 6, 9, and 10, the IDU 52 includes a plurality (e.g., pair) of power supply contacts 170 and a plurality of communication contacts 172 (FIG. 6), which in turn, couple to a plurality of passthrough power contacts 180 and a plurality of passthrough communication contacts 182 of the SIM 43. The generator 200 includes input power contacts 280 and pass-through communication contacts 282, which are configured to couple to the contacts 170 and 172 of the IDU 52 through the contacts 180 and 182 of the SIM 43, respectively. The instrument 50 also includes a plurality of electrosurgical contacts 190 and a plurality of communication contacts 192 (FIG. 6), configured to couple to a plurality of output power contacts 284 and the pass-through communication contacts 282 of the generator 200. Thus, the generator 200 provides a communication pathway between the IDU 52 and the SIM 43 at the proximal end and the instrument 50 at the distal end. In embodiments, the SIM 43 may be omitted and the IDU 52 may be coupled directly to the generator 200. A sterile drape may be coupled to the robotic arm 40 using any other suitable means.

The generator 200 also receives input power from the IDU 52 and outputs electrosurgical radiofrequency (RF) energy. The electrosurgical generator 200 is a miniaturized electrosurgical generator and includes circuit components that are designed to produce high-frequency electrical current and deliver it to tissue in order to perform surgical procedures. The electrosurgical generator 200 includes a power supply, which provides the electrical power needed to operate the generator. The power supply may include a transformer, rectifier, and filter circuit that converts the input power into the appropriate voltage and current levels required by the generator. The electrosurgical generator 200 also includes a high-frequency oscillator, which generates the high-frequency electrical current that is used to perform the surgical procedures. The high-frequency oscillator may include an oscillator circuit, which produces AC at a specific radiofrequency, e.g., 480 kHz, and a power amplifier, which boosts the output of the oscillator to a desired level.

The electrosurgical generator 200 may be controlled through surgeon console 30 or the control tower 20. This allows the user to adjust the settings of the electrosurgical generator 200, such as the output power, intensity, or amplitude settings, select a desired mode (e.g., coagulate, vessel sealing, fulgurate, cut, blend, etc.). The controls may be embodied in a graphical user interface representing buttons, switches, slides, and dials that the user can use to adjust the generator's settings. In embodiments, the electrosurgical generator 200 may be activated through one of the foot pedals 36.

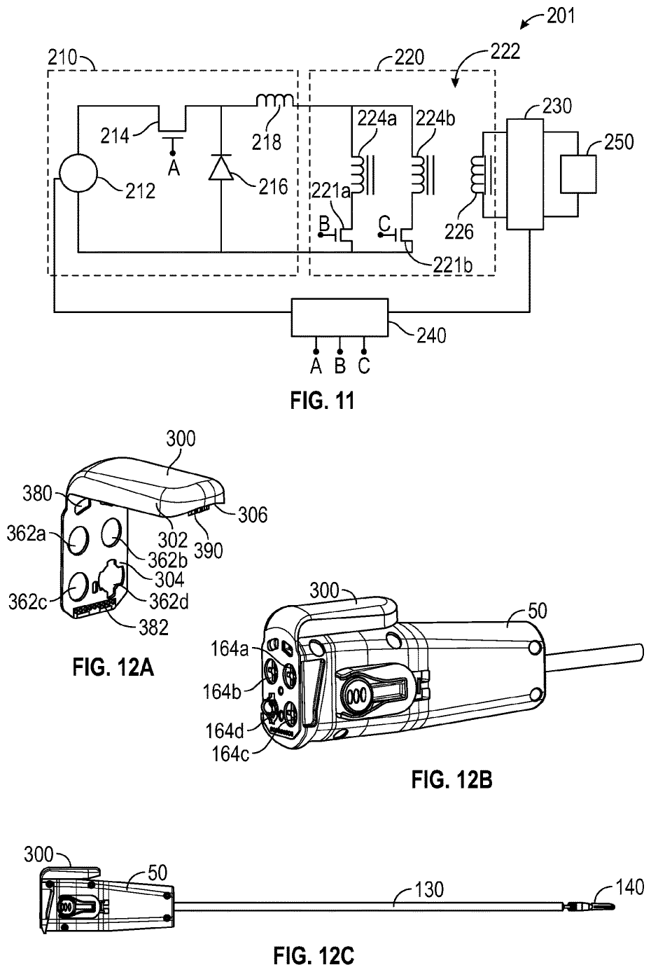

The electrosurgical generator 200 may have any suitable power architecture and topology suitable for generating electrosurgical energy and a generator circuit 201 of FIG. 11 is exemplary. The generator circuit 201 is configured as a current source. In embodiments, the generator circuit 201 may be configured as a voltage source. The generator circuit 201 has a buck converter 210 and an RF stage 220. Buck converter 210 is a switched mode power supply that may use two switches (e.g., a transistor and a diode). Buck converter 210 is coupled a voltage source 212, which may be DC power provided by the IDU 52 through input power contacts 180. Buck converter 210 includes a field effect transistor (FET) 214, diode 216 and an inductor 218. The buck converter alternates between connecting inductor 218 to voltage source 212 using FET 214 and diode 216 to store energy in inductor 218 and discharge energy from inductor 218 into the load.

RF stage 220, i.e., the high-frequency oscillator, includes a transformer 222 having primary windings 224a and 224b and secondary winding 226. Primary windings 224a and 224b are coupled to FETs 221a and 221b, respectively. Secondary winding 226 of transformer 222 outputs RF energy to a pair of output power contacts 284. The turns ratio for transformer 222 may be varied to limit the maximum voltage output of secondary winding 226. The generator circuit 201 may include a controller 240 that includes a microprocessor operably connected to a memory, which may be volatile type memory (e.g., RAM) and/or non-volatile type memory. The controller 240 includes an output port that is operably connected to FETs 214, 221a and 221b allowing the controller 240 to control the output of the generator circuit 201 according to either open and/or closed control loop schemes. Those skilled in the art will appreciate that the microprocessor may be substituted by any logic processor or analog circuitry (e.g., control circuit) adapted to perform the calculations discussed herein.

The generator circuit 201 may implement closed and/or open loop control schemes that include a sensor circuit 230 having a plurality of sensors measuring a variety of tissue and energy properties (e.g., tissue impedance, tissue temperature, output current and/or voltage, etc.), and providing feedback to the controller 240. A current sensor can be disposed at either the active or return current path or both and voltage can be sensed at the active electrode(s). The controller 240 then transmits appropriate signals to control the output of generator circuit 201. The controller 240 also receives input signals from the input controls of the generator or the instrument. The controller 240 utilizes the input signals to adjust power output by the generator circuit 201 and/or performs other control functions thereon.

The sensor circuit 230 measures the electrical current (I) and voltage (V) supplied by transformer 222 in real time to characterize the electrosurgical process during both the matching sinusoidal and non-sinusoidal durations for a predetermined sampling period, the former being of short duration (e.g., half a cycle) and the latter being of long duration (e.g., about 15 cycles). This allows for the measured electrical properties to be used as dynamic input control variables to achieve feedback control. The current and voltage values may also be used to derive other electrical parameters, such as power (P=V*I) and impedance (Z=V/I). The sensor circuit 230 also measures properties of the current and voltage waveforms and determines the shape thereof.

FIGS. 12A-C, 13A, and 13B show an electrosurgical generator 300 according to another embodiment of the present disclosure. The electrosurgical generator 300 is substantially similar to the electrosurgical generator 200 with respect to electrosurgical output functionality and only mechanical differences are described. The electrosurgical generator 300 includes a housing 302 having a proximal plate 304 configured to engage with the SIM 43 and a bottom surface 306 (FIGS. 12A-C) configured to engage the instrument 50 as shown in FIGS. 13A and 13B. The plate 304 includes a plurality of openings 362a-d allowing the couplers 160a-d of the SIM 43 to engage the couplers 164a-d of the instrument 50. In embodiments, the SIM 43 may be omitted and the IDU 52 may be coupled directly to the instrument 50.

The distal plate 304 also includes power contact openings 380 and communication contact openings 382, which are configured to couple the contacts 190 and 192 of the instrument 50 to the contacts 170 and 172 of the IDU 52 and/or through the contacts 180 and 182 of the SIM 43. The bottom surface 306 of the generator 300 includes an electrical connection interface 390 (FIG. 12A) including a plurality of electrical contacts for receiving electrical power from the IDU 52 and output RF energy to the instrument 50 as well as for transmitting control signals to and from the IDU 52 and the instrument 50.

FIGS. 14A and 14B show an electrosurgical generator 400 according to a further embodiment of the present disclosure. The electrosurgical generator 400 is substantially similar to the electrosurgical generator 200 with respect to electrosurgical output functionality and only mechanical differences are described. The electrosurgical generator 400 includes a housing 402 having a proximal surface 404 and a bottom surface 406 configured to engage the instrument 50 as shown in FIGS. 14A and 14B. In this embodiment, the instrument 50 may be coupled directly to the SIM 43 and/or the IDU 52. Thus, the contacts 190 and 192 of the instrument 50 are coupled directly to the contacts 170 and 172 of the IDU 52 and/or the contacts 180 and 182 of the SIM 43.

The generator 400 may include a plurality of teeth 408 extending from the bottom surface 406. The plurality of teeth 408 are configured to engage the housing 120 of the instrument 50. The housing 120 may include a plurality of corresponding slits to engage the teeth 408. The generator 400 also includes an electrical connection interface 490 including a plurality of electrical contacts for receiving electrical power from the IDU 52 and output RF energy to the instrument 50 as well as for transmitting control signals to and from the IDU 52 and the instrument 50. The generator 400 may be coupled to the housing 120 of the instrument 50 by initially inserting the teeth 408 into the housing 120 (i.e., step 1) and then sliding the generator 400 proximally to engage the teeth 408 to the housing 120 (i.e., step 2). The interface 490 is also inserted into a connector (not shown) disposed in the housing 120.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical robotic system comprising:
an instrument drive unit including at least one motor and at least one power supply contact;
an electrosurgical generator configured to couple to the instrument drive unit, the electrosurgical generator includes a generator circuit configured to couple to the at least one power supply contact and to generate electrosurgical energy; and
an electrosurgical instrument configured to couple to the instrument drive unit and the electrosurgical generator, the electrosurgical instrument is actuatable by the instrument drive unit and energizable by the electrosurgical generator.

2. The surgical robotic system according to claim 1, wherein the electrosurgical instrument is selected from the group consisting of a monopolar scissors, an electrocautery hook, an electrocautery spatula, an electrocautery blade, a bipolar forceps, and a vessel sealer.

3. The surgical robotic system according to claim 1, wherein the electrosurgical instrument includes at least one instrument coupler configured to engage the at least one motor.

4. The surgical robotic system according to claim 3, wherein the electrosurgical generator includes at least one generator coupler configured to engage the at least one motor and the at least one instrument coupler.

5. The surgical robotic system according to claim 4, wherein the instrument drive unit further includes a plurality of first communication contacts, the electrosurgical generator includes a plurality of generator passthrough communication contacts, and the electrosurgical instrument includes a plurality of second communication contacts, such that the electrosurgical instrument is in electrical communication with the instrument drive unit.

6. The surgical robotic system according to claim 5, further comprising a sterile interface module configured to couple in-between the instrument drive unit and the electrosurgical generator.

7. The surgical robotic system according to claim 6, wherein the sterile interface module includes at least one interface coupler configured to interconnect the at least one generator coupler and the at least one instrument coupler.

8. The surgical robotic system according to claim 7, wherein the sterile interface module includes a plurality of interface passthrough communication contacts configured to provide electrical communication between the instrument drive unit and the electrosurgical instrument through the sterile interface and electrosurgical generator.

9. The surgical robotic system according to claim 1, wherein the generator circuit is at least one of a current source or a voltage source.

10. The surgical robotic system according to claim 1, further comprising:
a robotic arm configured to support the instrument drive unit and the electrosurgical instrument.

11. A surgical robotic system comprising:
an instrument drive unit including at least one motor and at least one power supply contact;
an electrosurgical instrument including an instrument housing configured to couple to the instrument drive unit, the electrosurgical instrument is actuatable by the instrument drive unit; and
an electrosurgical generator configured to couple to the instrument housing of the electrosurgical instrument and to receive electrical input power from the at least one power supply contact through the electrosurgical instrument, the electrosurgical generator includes a generator circuit configured to generate electrosurgical energy from the input power to energize the electrosurgical instrument.

12. The surgical robotic system according to claim 11, wherein the electrosurgical instrument is selected from the group consisting of a monopolar scissors, an electrocautery hook, an electrocautery spatula, an electrocautery blade, a bipolar forceps, and a vessel sealer.

13. The surgical robotic system according to claim 11, wherein the electrosurgical instrument includes at least one instrument coupler configured to engage the at least one motor.

14. The surgical robotic system according to claim 13, wherein the electrosurgical generator includes a generator housing having a proximal plate configured to be disposed between the instrument drive unit and the electrosurgical instrument.

15. The surgical robotic system according to claim 14, wherein the proximal plate includes at least one opening to allow for engagement between at least one instrument coupler and the at least one motor.

16. The surgical robotic system according to claim 11, further comprising:
a robotic arm configured to support the instrument drive unit and the electrosurgical instrument.

17. The surgical robotic system according to claim 11, wherein the generator circuit is at least one of a current source or a voltage source.

18. The surgical robotic system according to claim 11, wherein the instrument drive unit further includes a plurality of first communication contacts and the electrosurgical instrument includes a plurality of second communication contacts, such that the electrosurgical instrument is in electrical communication with the instrument drive unit.

19. The surgical robotic system according to claim 11, wherein the electrosurgical generator includes a plurality of teeth extending from the generator housing and configured to engage the instrument housing.

20. A surgical robotic system comprising:
an instrument drive unit including a plurality of motors, a plurality of power supply contacts, and a plurality of communication contacts;
an electrosurgical generator configured to couple to the instrument drive unit, the electrosurgical generator including:
a generator circuit configured to couple to the plurality of power supply contacts and to generate electrosurgical energy through a plurality of output power contacts;
a plurality of generator couplers configured to engage the plurality of motors; and
a plurality of generator passthrough communication contacts; and
an electrosurgical instrument configured to couple to the electrosurgical generator, the electrosurgical instrument is actuatable by the instrument drive unit and energizable by the electrosurgical generator, the electrosurgical instrument including:
a plurality of electrosurgical contacts configured to electrically couple to the plurality of output power contacts; and
a plurality of instrument couplers configured to engage the plurality of generator couplers.
